(19) United States
(12) Patent Application Publication
(10) Pub. No.: US 2005/0233354 A1
(43) Pub. Date: Oct. 20, 2005

(54) Genotyping degraded or mitochandrial DNA samples

(75) Inventor: Giulia C. Kennedy, San Francisco, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/042,920

(22) Filed: Jan. 24, 2005

Related U.S. Application Data
(60) Provisional application No. 60/538,750, filed on Jan. 22, 2004.

(51) Int. Cl. C12Q 1/68; C12P 19/34

(52) U.S. Cl. 435/6; 435/91.2

(57) ABSTRACT

Methods, arrays and kits for amplifying and analyzing nucleic acid from compromised biological samples are provided. A method for amplifying both nuclear and mitochondrial DNA from biological samples and for detecting sequences that are characteristic of the sample are disclosed. Samples are fragmented with a restriction enzyme, ligated to an adaptor and adaptor-ligated fragments are amplified. The amplified fragments are analyzed by hybridization to an array comprising probes to detect known variants in mitochondrial DNA. The array may also include probes to detect known polymorphisms in nuclear DNA. The methods are particularly useful for forensic analysis.

### FIELD OF THE INVENTION

The methods of the invention relate generally to amplification of nucleic acid and analysis of genotype from samples that are compromised and may be partially degraded.

### SUMMARY OF THE INVENTION

Methods and arrays for analysis of biological samples that may be partially degraded are disclosed. The methods include a method for amplifying mtDNA sequences after fragmentation with a restriction enzyme, ligation to adaptors and amplification using one or a few common primers. The amplified fragments are labeled, for example, using fluorescent or chemiluminescent labels and hybridized to an array of probes. Hybridization patterns are analyzed using computer systems and genotypes that are characteristic of the sample are detected. The genotype information may be used to identify a sample as coming from a particular source or to rule out a source. The amplification methods do not require locus specific priming. Combination arrays that have probes to mtDNA and to nuclear DNA polymorphisms are disclosed. The arrays may combine resequencing probe sets for mtDNA, genotyping probe sets for mtDNA polymorphisms and probe sets for genotyping nuclear polymorphisms.

### BRIEF DESCRIPTION OF THE FIGURE

FIG. 1 shows a flow diagram for “Mitochondrial and Compromised DNA Profiling”, or MCP, which is an unbiased method designed to amplify DNA fragments in degraded samples.

FIG. 2A shows a schematic of a resequencing array tiling strategy.

FIG. 2B shows a schematic of how genotyping array probe sets may be designed.

### DETAILED DESCRIPTION OF THE PREFERRED EMBODIMENTS

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT applications Nos. PCT/JUS99/00730 (International Publication Number WO 99/36760) and PCT/JUS01/04285, which are all incorporated herein by reference in their entirety for all purposes. See also, Fodor et al., Science 251(4995), 767-73, 1991, Fodor et al., Nature 364(6437), 555-6, 1993 and Pease et al. PNAS USA 91(11), 5022-6, 1994 for methods of synthesizing and using microarrays.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.). Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods are shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Additional methods of genotyping, complexity reduction and nucleic acid amplification are disclosed in U.S. patent application Ser. Nos. 60/508,418, 60/468,925, 60/493,085, 09/920,491, 10/442,021, 10/654,281, 10/316,811, 10/646,674, 10/272,155, 10/681,773 and 10/712,616 and U.S. Pat. No. 6,582,938. For additional information on genotyping methods see, for example, Kwok, P. Y. (2001), Annu Rev Genomics Hum Genet 2: 235-58 and Syvanen, A. C. (2001), Nat Rev Genet 2(12): 930-42. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Res. 11, 1418 (2001), in U.S. Pat. Nos. 6,361,947, 6,391,592 and U.S. Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 60/364,731 and in PCT application PCT/JUS99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/063,559 (United States Publication No. US20020183936), 60/349,546, 60/376,003, 60/394,574 and 60/403,381.

b) Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

“Biopolymer or biological polymer” is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. “Initiation Biomonomer” or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

“Complementary” refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

A “Combinatorial Synthesis Strategy” is an ordered strategy for parallel synthesis of diverse polymer sequences by sequential addition of reagents which may be represented by a reactant matrix and a switch matrix, the product of which is a product matrix. A reactant matrix is a I column by m row matrix of the building blocks to be added. The switch matrix is all or a subset of the binary numbers, preferably ordered, between I and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

A genome is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

The term “chromosome” refers to the heredity-bearing gene carrier of a living cell which is derived from chromatin and which comprises DNA and protein components (especially histones). The conventional internationally recognized individual human genome chromosome numbering system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ bp while the smallest chromosome, chromosome no. 22, contains about 5.3×10⁷ bp.

A chromosomal region is a portion of a chromosome. The actual physical size or extent of any individual chromosomal region can vary greatly. The term region is not necessarily definitive of a particular one or more genes because a region need not take into specific account the particular coding segments (exons) of an individual gene.

An allele refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”. At each autosomal specific chromosomal location or “locus” an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. patent application Ser. No. 08/630,427, filed Apr. 3, 1996.

The term hybridization refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example tow complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature and the concentration of salt in the reaction. Hybridizations are usually performed under stringent conditions, for example, at a salt concentration of no more than 1 M and a temperature of at least 25° C. For example, conditions of 5×SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for allele-specific probe hybridizations. In a particularly preferred embodiment hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual.

A “ligand” is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

“Linkage disequilibrium or allelic association” refers to the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. Linkage disequilibrium may be used to identify markers that are associated with a phenotype, such as a disease, even though the marker may have no contribution to the phenotype. For example, marker A may be linked to gene X which carries a mutation that contributes to a disease phenotype but may not have been identified or may not be readily detectable. Detection of marker A may be used to assess susceptibility to the disease. Linkage disequilibrium may also be used to identify genes that contribute to phenotypes or genomic regions suspected of containing genes that contribute to phenotypes.

“Mixed population” or “complex population” refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

“mRNA” or “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

“Nucleic acids” according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Oligonucleotides may be chemically synthesized and may include modifications. Amino modifier reagents may be used to introduce a primary amino group into the oligo. A primary amino group is useful for a variety of coupling reactions that can be used to attach various labels to the oligo. The most frequently used labels are in the form of NHS-esters, which can couple with primary amino groups. A variety of derivatives of biotin are available in which the biotin moiety is connected (through the 4-carboxybutyl group) to a linker molecule that can be attached directly to an oligonucleotide. Fluorescent dies such as 6-FAM, HEX, TET, TAMRA, and ROX may be coupled to an oligo. Phosphate groups may be attached to the 5′ and/or 3′ end of an oligo. Oligos may also be phosphorothioated. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified “backbone” of an S-Oligo is resistant to the action of most exonucleases and endonucleases. In some embodiments the oligo is sulfurized only at the last few residues at each end of the oligo. This results in an oligo that is resistent to exonucleases, but has a natural DNA center. Degenerate bases may also be incorporated into an oligo may also be incorporated into an oligo Additional modifications that are available include, for example, 2′O-Methyl RNA, 3′-Glyceryl, 3′-Terminators, Acrydite, Cholesterol labeling, Dabcyl, Digoxigenin labeling, Methylated nucleosides, Spacer Reagents, Thiol Modifications Deoxylnosine, DeoxyUridine and halogenated nucleosides.

A “probe” is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. Arrays comprising all possible probes sequences of a given length are disclosed in U.S. Pat. No. 6,582,908.

A “primer” is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

“Polymorphism” refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

A “target” is a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

“Restriction enzyme” or “restriction endonuclease” in general recognizes a specific nucleotide sequence of four to eight nucleotides and cuts the DNA at a site within or a specific distance from the recognition sequence. A number of methods disclosed herein require the use of restriction enzymes to fragment the nucleic acid sample. For example, the restriction enzyme EcoRI recognizes the sequence GAATTC and will cut a DNA molecule between the G and the first A. The length of the recognition sequence is roughly proportional to the frequency of occurrence of the site in the genome. A simplistic theoretical estimate is that a six base pair recognition sequence will occur once in every 4096 (4⁶) base pairs while a four base pair recognition sequence will occur once every 256 (4⁴) base pairs. In silico digestions of sequences from the Human Genome Project show that the actual occurrences may be more or less frequent, depending on the sequence of the restriction site. Because the restriction sites are rare, the appearance of shorter restriction fragments, for example those less than 1000 base pairs, is much less frequent than the appearance of longer fragments. Many different restriction enzymes are known and appropriate restriction enzymes can be selected for a desired result. (For a description of many restriction enzymes and recommended reaction conditions see, New England BioLabs Catalog which is herein incorporated by reference in its entirety for all purposes).

“Adaptor sequences” or “adaptors” are generally oligonucleotides of at least 5, 10, or 15 bases and preferably no more than 50 or 60 bases in length; however, they may be even longer, up to 100 or 200 bases. Adaptor sequences may be synthesized using any methods known to those of skill in the art. For the purposes of this invention they may, as options, comprise primer binding sites, recognition sites for endonucleases, common sequences and promoters. The adaptor may be entirely or substantially double stranded. A double stranded adaptor may comprise two oligonucleotides that are at least partially complementary. The adaptor may be phosphorylated or unphosphorylated on one or both strands. Adaptors may be more efficiently ligated to fragments if they comprise a substantially double stranded region and a short single stranded region which is complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible. Blunt ends can be converted to sticky ends using the exonuclease activity of the Klenow fragment. For example when DNA is digested with PvuII the blunt ends can be converted to a two base pair overhang by incubating the fragments with Klenow in the presence of dTTP and dCTP. Overhangs may also be converted to blunt ends by filling in an overhang or removing an overhang.

Methods of ligation will be known to those of skill in the art and are described, for example in Sambrook et at. (2001) and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E.coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′→5′ phosphodiester bond, substrates include single-stranded RNA and DNA as well as dinucleoside pyrophosphates; or any other methods described in the art.

When a fragment has been digested on both ends with the same enzyme or two enzymes that leave the same overhang, the same adaptor may be ligated to both ends. Digestion with two or more enzymes can be used to selectively ligate separate adaptors to either end of a restriction fragment. For example, if a fragment is the result of digestion with EcoRI at one end and BamHI at the other end, the overhangs will be 5′-AATT-3′ and 5′GATC-3′, respectively. An adaptor with an overhang of AATT will be preferentially ligated to one end while an adaptor with an overhang of GATC will be preferentially ligated to the second end.

An adaptor may be ligated to one or both strands of the fragmented DNA. In some embodiments a double stranded adaptor is used but only one strand is ligated to the fragments. Ligation of one strand of an adaptor may be selectively blocked. Any known method to block ligation of one strand may be employed. For example, one strand of the adaptor can be designed to introduce a gap of one or more nucleotides between the 5′ end of that strand of the adaptor and the 3′ end of the target nucleic acid. Adapters can be designed specifically to be ligated to the termini produced by restriction enzymes and to introduce gaps or nicks. For example, if the target is an EcoRI digested fragment an adapter with a 5′ overhang of TTA could be ligated to the AATT overhang left by EcoRI to introduce a single nucleotide gap between the adaptor and the 3′ end of the fragment. Phosphorylation and kinasing can also be used to selectively block ligation of the adaptor to the 3′ end of the target molecule. Absence of a phosphate from the 5′ end of an adaptor will block ligation of that 5′ end to an available 3′OH. For additional adaptor methods for selectively blocking ligation see U.S. Pat. No. 6,197,557 and U.S. Ser. No. 09/910,292 which are incorporated by reference herein in their entirety for all purposes.

“Mitochondria” are subcellular organelles that contain an extrachromosomal genome (mtDNA) distinct from the nuclear genome. A mitochondrion contains between 2 and 10 copies of mtDNA and a somatic cell may have as many as 1000 mitochondria. A single cell may have as many as 10,000 copies of mtDNA compared to only 2 copies of the nuclear genome. In humans mtDNA is an approximately 16,569 base pair circular molecule which has a higher mutation rate than the nuclear genome, resulting from low fidelity of mtDNA polymerase and the apparent lack of mtDNA repair mechanisms. Some regions of the mtDNA exhibit an evolution rate that is 5-10 times that of single-copy nuclear genes. Hypervariable regions of the mtDNA have been identified within the control region of mtDNA, an apoproximately 1,100 base pair non-coding region. Two regions, hybervariable region 1 (HV1) and hypervariable region 2 (HV2) have been used for human identity testing because of the large amount of variation found in these regions. HV1 and HV2 span approximately from position 16,024 to 16,365 and position 73 to 340, respectively and account for an average of 8 differences between Caucasian individuals and 15 differences between individuals of African descent. (See, Budowle et al. Forensic Sci. Int. 103:23-35 (1999) and Vigilant et al. Science 253:1503-7 (1991). HV1 and HV2 are routinely used for forensic testing purposes (see, Budowle et al. (2003)).

Unlike nuclear DNA, mtDNA is maternally inherited and is not subject to recombination. In the absence of mutation, the mtDNA sequence of siblings and all maternal relatives is identical. This feature of mtDNA makes it particularly well suited for forensic analysis. For example, mtDNA from unidentified human remains can be analyzed and compared to reference samples of maternal relatives of missing persons. If the unknown sequence or hybridization pattern matches the maternal relative a determination of the identity of the remains can be made. An unknown sample may be compared to samples from suspected relatives and if the sequence matches a determination of relatedness may be made with a high degree of certainty.

“Heteroplasmy” The human body contains trillions of cells, each of which can contain thousands of copies of the mtDNA genome. Complete homoplasmy (the same sequence of mtDNA) for each of these mtDNA molecules would be surprising because of the immense amounts of mtDNA present in the body. Thus, heteroplasmy is expected to be present at some level in most, if not all, individuals. Heteroplasmy is the occurrence of more than one sequence at a particular position in a DNA sequence, and there are two forms of heteroplasmy found in mtDNA. Sequence heteroplasmy, or point heteroplasmy, is the occurrence of more than one base at a particular position or positions in the mtDNA sequence. Length heteroplasmy is the occurrence of more than one length of a stretch of the same base in a mtDNA sequence. Much heteroplasmy is probably present at levels that are lower than can be detected by current methods so heteroplasmy is used as an operational term used when the current scientific methods are capable of detecting more than one sequence in an individual.

Heteroplasmy was first observed in forensic mtDNA sequences in 1994 by the Forensic Science Service (FSS) in the United Kingdom while identifying the remains of the Romanov family (Gill et al. Nature Genetics 6:130-135, (1994) and Ivanov, P. L., M. J. Wadhams, et al. (1996), Nat Genet. 12(4): 417-20. Heteroplasmy may be used to enhance confidence in a predicted match. When heteroplasmy is observed at the same position in an unknown and a reference sample and all of the other bases are identical, the significance of the match is enhanced.

Because of differences in the mechanisms by which cells are generated in different tissues, heteroplasmy rates may vary depending on the tissue type. For example, a hair sample may contain mostly C at a particular position in the mtDNA genome, while a blood sample from the same individual may contain equal amounts of C and T at the same position. If different tissues demonstrate heteroplasmy with the presence of common bases at every position, then a sequence concordance is present, and there is a higher probability that the two samples came from the same source or maternal lineage. In cases where heteroplasmy is observed, additional known samples can be analyzed to determine if the heteroplasmy is observed in other tissues. DNA profiling refers to the general use of DNA tests to establish identity or relationships. DNA profiling may be performed by a number of methods including, for example, minisatellite probes, microsatellite markers, y-chromosome polymorphism analysis and mtDNA polymorphism analysis. DNA profiling may be used for a variety of applications including, for example, determining the zygosity of twins, disproving or establishing paternity, and for forensic investigation, for example, identification of missing persons and identification of a criminal. For additional information on the use of forensic DNA evidence see, Evett, I W and Weir, B S (1998), Interpreting DNA Evidence: Statistical Genetics for Forensic Scientists, Sinauer. For a review of genetic methods see, for example, Strachan, T and Read, A P (2004). Human Molecular Genetics 3. Garland Science.

“Simple tandem repeats”, or “STRs”, are useful markers for scoring human genetic variation and are the mainstay in forensic identity testing (reviewed in Gill Biotechniques 32(2): 366-8, 370, 372, 2002). STR analysis requires PCR amplification by sequence-specific primers followed by size discrimination on a gel-based platform. A panel composed of 13 or 16 STRs is used extensively in forensic science for identity matching between test and reference sample. Commercial kits for analysis of STR loci are available from, for example, Promega, Madison, Wis. The amplification of all loci is performed in one reaction as a multiplex PCR. One limitation of such a system is that the addition of more markers (for example to add ancestry-informative markers, to include mitochondrial or plant DNA sequences) would require re-optimization of the multiplex PCR reaction. While high levels of STR multiplexing by PCR (100-1000 fold) are achievable with extensive optimization, this approach is difficult to scale to large numbers of loci, due to limited space on the gel for resolving additional fragments. A further bottleneck is that individual profiles must be examined and checked by highly-trained personnel and often reviewed by a second individual because of stutter peaks and sizing reproducibility that confound interpretation of results.

The “F_{ST} statistic” may be used to identify SNPs that are ancestry-informative markers (AIMs), F_{ST} is an estimate of the geographic structure between two populations, for each SNP. F_{ST} values vary from 0 to 1; as allele frequency differences between populations become more pronounced, F_{ST} values increase. When calculating 0.061, 0.094 and 0.065 for SNPs in an African-American versus Caucasian population, African-American versus Asian populations and Caucasian versus Asian populations the mean F_{ST} values are typically less than 0.1 indicating that the majority of markers show very small inter-population frequency differences. However, there is a subset of SNPs whose allele frequencies differ significantly in one population versus the other two. These SNPs, called ancestry-informative markers, or AIMs, can be used to map complex diseases using admixture-generated linkage disequilibrium, or MALD. See Collins-Schramm, H. et al., Am. J. Hum. Genet. 70, 737-750 (2002), Briscoe, D. et al., J. Hered. 85, 59-63 (1994), Parra, E. J. et al., Am. J. Hum. Genet. 63, 1839-1851 (1998), and McKeigue, P. M. et al., Ann. Hum. Genet. 64, 171-186 (2000) each of which is incorporated herein by reference in its entirety.

C. Genotyping Degraded or Mitochondrial DNA Samples

Mitochondrial DNA is a circular DNA present in cells at high copy number, hence it is often used in identification of samples where there is extensive degradation of genomic DNA. For example, mitochondrial DNA has been extracted from ancient Neanderthal remains, amplified and analyzed by sequencing (Ovchinnikov et al., Nature 404(6777): 490-3, 2000. Furthermore, the criminal justice system makes extensive use of mitochondrial DNA testing to identify crime scene evidence containing badly degraded nuclear DNA samples. The primary mode of amplification has been PCR with sequence-specific oligonucleotide primers. For a review of the use of mitochondrial DNA in forensic applications, see, for example, Budowle B. et al., Annu Rev Genomics Hum Genet.; 4: 119-41, 2003. See also, United States Department of Justice, Office of Justice Programs, National Institute of Justice (2002), “Using DNA to Solve Cold Cases” and United States Department of Justice, Office of Justice Programs, National Institute of Justice (2003), “Report to the Attorney General on Delays in Forensic DNA Analysis.”

Locus specific PCR on degraded samples can result in failure of amplification of some sequences if degradation has occurred at or between the primer sites, resulting in loss of the information for that amplicon. High-throughput target preparation and analysis strategies that capture nucleotide sequence information from the sample using an amplification method that is not biased toward specific targets are disclosed.

The features of mitochondrial DNA (mtDNA) that make it useful for forensics include: high copy number, lack of recombination, and matrilineal inheritance. Typing of mtDNA is commonly used in forensic biology, for example, and to analyze old bones, teeth, hair shafts, and other biological samples where nuclear DNA content is low or of poor quality. For methods of analysis of mtDNA from hair shafts see Wilson, et al., BioTechniques (1995) 18:662-669 and Higuchi, et al. (1988), Nature 332: 543-546.

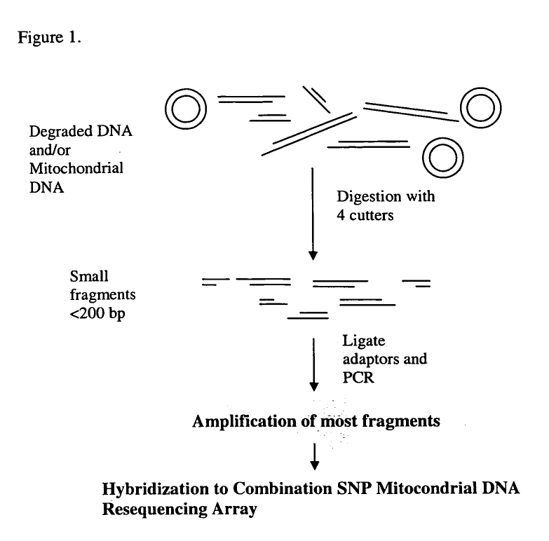

Samples in which DNA is degraded are often difficult to analyze by current methods using locus-specific PCR because of damage at or between primer binding sites. An alternative method that uses ligation of adapter sequences and amplification using common primers is disclosed. The method, Mitochondrial and Compromised DNA Profiling (MCP), may be used to amplify fragments of DNA that are present in the degraded samples nonspecifically and without limiting the choice of target, all sequences that are present are targets for amplification (see, FIG. 1). This is unlike locus specific amplification which is limited to the targets that are complementary to the selected primers; if that target is absent or degraded it will not be amplified and will not be detected in subsequent analysis steps. The disclosed approach does not require use of a specific primer sequence in the target for amplification. All DNA, regardless of source, may be amplified. The amplified fragments may then be hybridized to an array to determine which SNPs and sequences are present. The array may include probes to analyze specific regions of interest, simplifying the analysis and interpretation. The MCP method begins by isolating DNA from a sample and subjecting it to controlled fragmentation by digestion with one or more restriction endonucleases. In a preferred embodiment restriction endonucleases with a 4-bp recognition site (4 cutters) are used. Each 4 cutter enzyme will recognize and cut DNA on average, every 256 bp. Digestion with 2 or more 4 cutters results in even smaller fragments. The size of the resulting fragment may be varied by varying the enzymes used. Many restriction enzymes cleave DNA to produce overhanging single stranded regions termed “sticky ends” which can be used to facilitate ligation of the fragment to an adaptor sequence with a complementary overhang. The fragments may be ligated with one or more adaptors that may have a region of common sequence that may be used as a common priming site for PCR amplification using a single generic primer. The ends of the fragments may be self complementary resulting in the formation of step loop structures that may amplify with reduced efficiency, especially with smaller fragment sizes. To overcome this effect high concentrations of primer may be used. See, U.S. patent application Ser. No. 09/916,135 for additional discussion.

There are a number of restriction enzymes that have a 4 base pair recognition sequence. Examples include, Aci I, Alu I, Bfa I, BstU I, CviA II, Hae III, Hha I, Hpa II, Mse I, Msp I, Sau3A I, Dpn II, Mbo I, Fat I, Nla III, and Tsp509 I. Characteristics of individual enzymes may make them more or less suited for use in one or more embodiments. For example, an enzyme that generates a 4 base overhang, such as Sau3A I, may be preferred in some embodiments over an enzyme that generates a 2 base overhang, such as Mse I, or an enzyme that generates blunt ends, such as Alu I. The 4 base overhang may result in more efficient ligation to an adaptor sequence than a 2 base overhang or a blunt end.

In one aspect the sample may be digested with two or more 4 cutters that generate distinct overhangs. In one aspect an enzyme with a 4 base pair recognition sequence is combined with an enzyme that has a 5, 6 or larger recognition sequence. Adaptors that are complementary to each of the overhangs are used for ligation so that at least some of the fragments will have a first adaptor ligated to one end and a second, different adaptor ligated to the other end. A pair of primers with one primer that is complementary to one adaptor sequence and a second primer that is complementary to the second adaptor sequence may be used for amplification.

In one aspect an adaptor that ligates to blunt ends may be included. Some fragments may be digested on one end by the restriction enzyme so that the sticky ended adaptor can be ligated to that end, but may lack a restriction site on the other end. Those fragments can be treated to generate blunt ends and ligated to a blunt ended adaptor. PCR amplification may be used to amplify those fragments. This may allow recovery of some fragments that would otherwise not be amplified.

Unlike amplification methods that are directed at reducing the complexity of a sample, for example the WGSA assay used in conjunction with the Affymetrix Mapping 10K array, MCP is intended to amplify as much of the remaining genetic material as possible. Because the resulting complexity is likely to be very low, high mass amounts of amplified target can be hybridized to the array to improve signal. The disclosed methods result in amplification of all DNA, regardless of source, including non-human, provided it can be digested with the selected enzymes. Despite this lack of amplification specificity, only desired SNPs and sequences will be analyzed and interrogated on the array, simplifying the analysis and interpretation. In some aspects the potential for cross hybridization between nuclear and mitochondrial DNA sequences, as well as cross hybridization between DNA of other species and human sequences on the array is taken into consideration and minimized by selecting probes that are less likely to result in cross hybridization or by using appropriate controls. Potential for cross hybridization may be determined by comparing a probe sequence to databases of genomic information for other organisms. In one embodiment probes to non-human sequences are included on the array to obtain more information about the sample.

Publicly available databases containing mtDNA polymorphism and sequence information may be used during the analysis of MCP results. See, for example, the mitomap web site at mitomap.org. The assay may be optimized using standard PCR methods to use as little starting DNA as possible and to test for the effects of common PCR inhibitors in forensic samples e.g. heme etc. In one embodiment MCP is performed using large format arrays (ie 169 format). In other embodiments smaller format arrays may be used to reduce costs and to facilitate HTA implementation.

MCP may be used to generate evidence in criminal proceedings so accuracy and robustness of information is needed in many embodiments. The disclosed methods use multiple data points to increase the confidence level of the interpretation. In some aspects the array can resequence all possible single base variants of human mtDNA. With a degraded sample it is expected that only a subset of the mtDNA polymorphisms will be successfully called, but the SNPs that can be called in one sample may be different from the SNPs that can be called in another sample, depending on the degradation. By providing a tool capable of sequencing all possible SNPs in mtDNA, bias in the assay toward a particular set of SNPs is minimized or eliminated.

Reference SNP genotypes may be generated using the Mapping10K Array and Assay (Affymetrix, Santa Clara) on nuclear DNA from control samples. In preferred embodiments the samples are mixtures of mtDNA and nuclear DNA. The samples may also be mixtures of DNA from two or more individuals. The assay may be used to characterize small amounts of starting DNA, for example, to use as little starting DNA as possible. In some embodiments the samples are tested for the presence of common contaminants in forensic samples e.g microbial, animal, or plant DNA, and heme for example.

In some embodiments samples may be analyzed on the 10K, 100K or resequencing (CustomSeq or Slingshot) arrays or other DNA analysis arrays available from Affymetrix, Inc. For additional information see the Affymetrix web site at Affymetrix.com. Additional methods of sample preparation and analysis are described in U.S. patent application Ser. Nos. 09/916,135, 10/681,773, 10/740,230, 10/316,629, 10/650,332, and 10/463,991 which are each incorporated herein by reference in their entireties for all purposes. Method and arrays for resequencing are described in U.S. patent application Nos. Ser. 10/843,527, 10/829,015, 10/028,482 and 10/658,879 and in Cutler, et al. (2001), Genome Res. 11(11): 1913-25 and Warrington, et al. (2002), Hum Mutat. 19(4): 402-9. Applications of arrays to forensic analysis are also disclosed in U.S. patent application No. 60/635,850 filed Dec. 13, 2004 and in Holland, M. M. and T. J. Parsons (1999), Forensic Sci Rev. 11: 21-50 and Anslinger, et al. (2001), Int J Legal Med 114(3): 194-6.

MtDNA analysis may be used, for example, where biological evidence may be degraded or small in quantity. Cases in which hairs, bones, or teeth are the only evidence retrieved from a crime scene are particularly well-suited to mtDNA analysis. Missing persons cases can benefit from mtDNA testing when skeletonized remains are recovered and compared to samples from the maternal relatives or personal effects of missing individuals. Also, hairs recovered at crime scenes can often be used to include or exclude individuals using mtDNA testing. Standard analysis procedures are known in the art, including a mtDNA population database, methods of analysis of mtDNA population statistics, and methods of assuring data quality. See Isenberg and Moore, Forensic Science Communications 1:2 (1999).

Mitochondrial DNA differs from nuclear DNA in its location, its sequence, its quantity in the cell, and its mode of inheritance. The nucleus of the cell contains two sets of 23 chromosomes—one paternal set and one maternal set. However, cells may contain hundreds to thousands of mitochondria, each of which may contain several copies of mtDNA. Nuclear DNA has many more bases than mtDNA, but mtDNA is present in many more copies than nuclear DNA. This characteristic of mtDNA is useful in situations where the amount of DNA in a sample is very limited. Typical sources of DNA recovered from crime scenes include hair, bones, teeth, and body fluids such as saliva, semen, and blood.

In humans, mitochondrial DNA is inherited from the mother (Case and Wallace Somatic Cell Genetics 7:103-108, 1981; Giles et al. Proceedings of the National Academy of Sciences, 77:6715-6719, 1980; Hutchison et al. Nature 251:536-538, 1974) although there are reports of paternal leakage. Thus, the mtDNA sequences obtained from maternally related individuals, such as a brother and a sister or a mother and a daughter, will exactly match each other in the absence of a mutation. This characteristic of mtDNA is advantageous in missing persons cases as reference mtDNA samples can be supplied by any maternal relative of the missing individual (Ginther et al., Nat Genet 2(2): 135-8, 1992; Holland et al., J Forensic Sci 38(3): 542-53, 1993; and Stoneking et al. Am J Hum Genet 48(2): 370-82.1991). However, mtDNA analysis is limited when compared to nuclear DNA analysis in that it cannot discriminate between individuals of the same maternal lineage. Discrimination may be provided by analysis of nuclear SNPs. An array combining detection of nuclear SNPs and mtDNA polymorphisms may be used to identify maternal lineage and then to discriminate between individuals within that lineage.

The human mtDNA genome is approximately 16,569 bases in length and has two general regions: the coding region and the control region. The coding region is responsible for the production of various biological molecules involved in the process of energy production in the cell. The control region is responsible for regulation of the mtDNA molecule. Two regions of mtDNA within the control region have been found to be highly polymorphic, or variable, within the human population (Greenberg et al., Gene 21:33-49, 1983). These two regions are termed Hypervariable Region I (HV1), which has an approximate length of 342 base pairs (bp), and Hypervariable Region II (HV2), which has an approximate length of 268 bp. Forensic mtDNA examinations are performed using these two regions because of the high degree of variability found among individuals.

Many current methods of forensic analysis of mtDNA sequence approximately 610 bp of mtDNA. By convention, human mtDNA sequences are described using the first complete published mtDNA sequence as a reference (Anderson et al. Nature 290: 457-465, 1981). This sequence is commonly referred to as the Anderson sequence. It is also called the Cambridge reference sequence or the Oxford sequence. Each base pair in this sequence is assigned a number. Deviations from this reference sequence are recorded as the number of the position demonstrating a difference and a letter designation of the different base. For example, a transition from A to G at Position 263 would be recorded as 263 G. If deletions or insertions of bases are present in the mtDNA, these differences are denoted as well.

Methods for amplifying genomic nucleic acid and for genotyping or sequencing regions of nucleic acid from samples containing degraded nuclear DNA or mitochondrial DNA are disclosed. Arrays comprising probes tiled to resequence mtDNA regions and to genotype nuclear SNPs are disclosed. The disclosed arrays and methods may be used to characterize samples of unknown origin. In some embodiments a plurality of the SNPs are ancestry informative SNPs. In one embodiment nucleic acids are obtained from a sample using standard methods and digested with at least one restriction enzyme, in a preferred embodiment a mixture of 2 or more 4-cutter restriction enzymes is used, the fragments are ligated to at least one adaptor, in a preferred embodiment the fragments are ligated to a plurality of adapters including adapters with sticky ends that are complementary to the ends left by the restriction enzyme(s) used and an adapter with blunt ends. The adapter ligated fragments are amplified by PCR using a common primer that is complementary to the adapters or using two or more primers that are each complementary to an adaptor sequence. The amplified fragments are labeled and hybridized to high-density microarrays (as described in Kennedy et al. Nat Biotechnol 21(10): 1233-7, 2003).

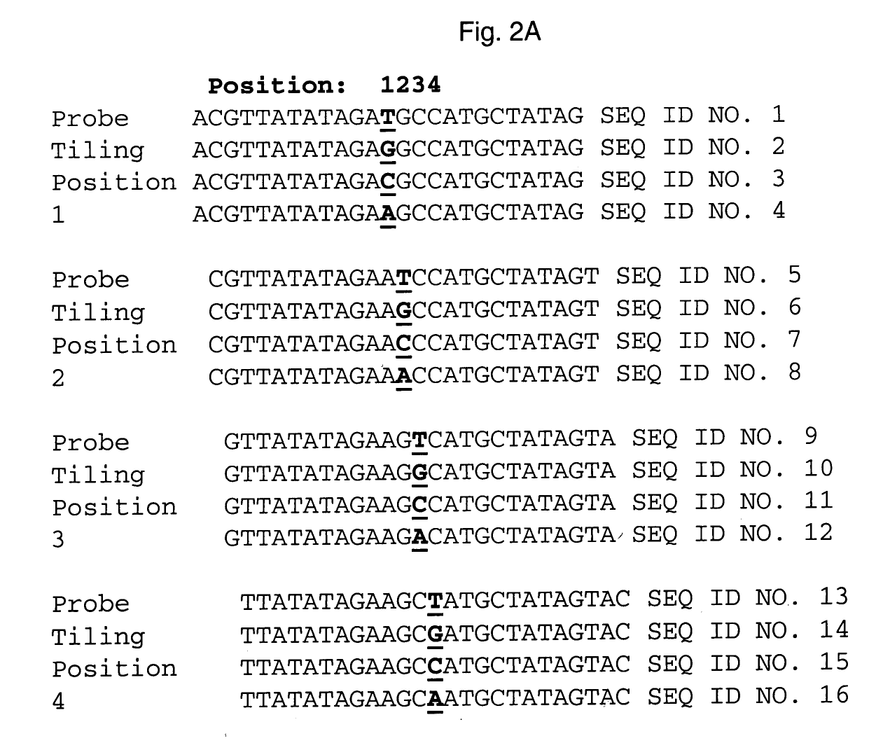

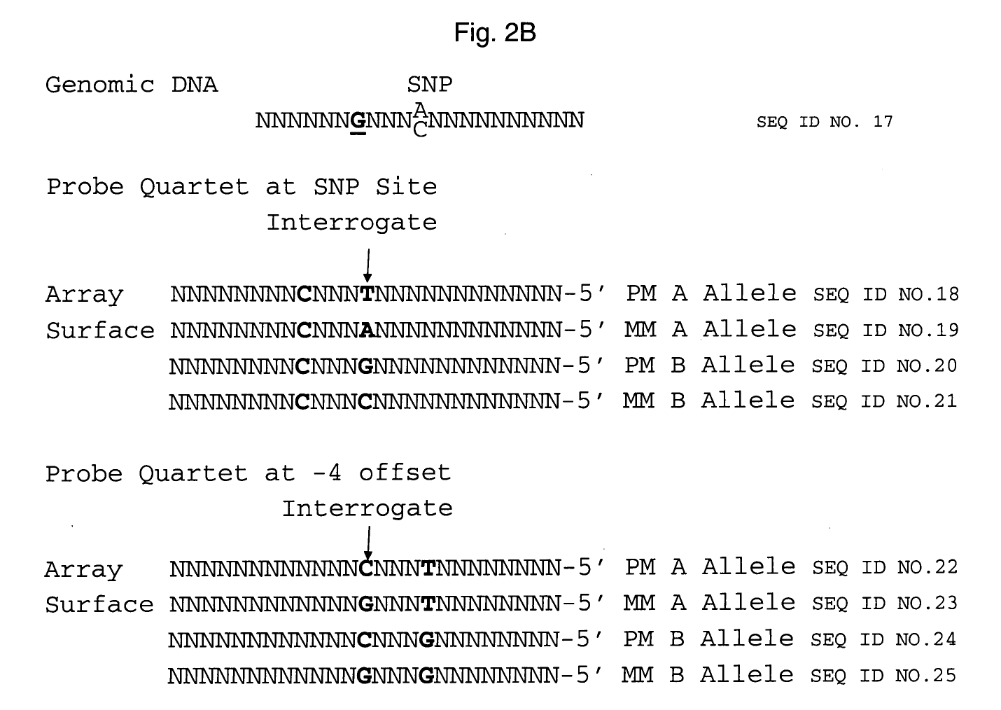

In one embodiment the amplified fragments may be hybridized to a resequencing array. Mitochondrial resequencing arrays are available from Affymetrix and have been used for a variety of analysis, Chee et al., Science 274(5287):610-4, 1996 and Maitra, et al., Genome Res. 14(5), 812-9, 2004. Resequencing arrays comprise tiled oligonucleotide probes to interrogate the sequence of individual positions in the target sequence. An exemplary tiling strategy is shown in FIG. 2A. For each position being interrogated the array includes at least four probes, each probe varying in the identity of the base at the interrogation position. SEQ ID NOs. 1-4 interrogate the first position, each having a different base at the interrogation position. There are four positions interrogated in the figure. Only the probes for one strand are shown, but a similar set of probes is typically included for the opposite strand. Typically the interrogation position is the central position of the probes, for example, position 13 of a 25 base probe. To detect variation in a target a probe set may be included on the array for each position in the target. For example, the GeneChip Mitochondrial Resequencing Array (P/N 510987) resequences more than 15,000 base pairs of the coding sequence from the human mitochondrial genome. Each 25-mer probe is varied at the central position to incorporate each possible nucleotide (A,C,G or T) on both strands. The array may be used to detect both novel and known SNPs in human mtDNA. The array may have probe sets to interrogate more than 1,000, 2,000, 3,000, 5,000, 10,000 or 15,000 different interrogation positions in mtDNA. A probe set is included for each interrogation position. The sequence to be interrogated may be contiguous or non-contiguous. In one aspect the sequence is non-contiguous and represents two or more different regions of mtDNA.

In another aspect a mitochondrial resequencing array that includes probes tiled for both the coding regions and the non-coding D-loop may be used. The D-loop is more variable than the coding regions of mtDNA and probes may be designed to accommodate this increased variability. For example, two polymorphisms may fall within the same 25 base probe so probe sets may be designed to interrogate the different combinations of the two polymorphisms. For example, if SNP1 is 5 bases from SNP2, the sequence of a perfect match probe for SNP2 will depend on the identity of SNP1. Probe sets to interrogate SNP2 may be designed for each of the possible alleles of SNP1.

In another embodiment the amplified fragments may be hybridized to a genotyping array. Genotyping arrays and methods of using genotyping arrays are described, for example, in Matsuzaki et al. Genome Res. 14:414-425 (2004) and John et al. Amer. Jour. Hum. Gen. 75:54-64, (2004). See also, Lipshutz, et al. (1999), Nat Genet. 21(1 Suppl): 20-4 and Liu, et al. (2003), Bioinformatics 19(18): 2397-403.

Genotyping arrays have allele specific probes that are perfectly complementary to specific polymorphisms. In some embodiments the array includes a probe set for each SNP in a pre-selected set of SNPs. A probe set includes perfect match probes and control probes for each allele of the SNP being interrogated. In one aspect the array has probe sets for each of a plurality of pre-selected mtDNA SNPs. In another aspect the array has probes for known mtDNA SNPs and probes for nuclear SNPs. SNPs may be selected for interrogation because of proximity to a restriction site for a selected restriction enzyme. SNPs may be selected for interrogation from the publicly available databases of human mtDNA polymorphisms.

FIG. 2B shows an example of a genotyping probe set. Seq ID Nos. 18-20 are the four probes for the SNP interrogation site. The SNP site is at position 13, the interrogation position. SEQ ID NOs. 22-25 represent the −4 probe set for this SNP. The interrogation position is −4 in relation to the SNP site. A genotyping probe sets may include, for example, probe sets for 0, −2, −4, +1 and +4 positions, relative to the SNP. Either or both strands may be interrogated.

In some aspects, particularly in a genomic region that is highly variable, such as the D-loop of mtDNA, probes that include variation in the sequence surrounding the SNP being interrogated may be used. For example, if there is a neighboring SNP that is within the probe region of the interrogation SNP, the SNP being interrogated, then variation in the neighboring SNP will effect the perfect complementarity of the probes to the interrogation SNP. Probe sets to interrogate the interrogation SNP may be included for each allele of the neighboring SNP. In one aspect variation in a neighboring SNP is analyzed in separate features, so that probes that are perfect match to each allele of the neighboring SNP are in different features of the array. In another aspect the probes for the interrogation SNP include variation to account for the neighboring SNP within the same feature, for example, the perfect match probe for the interrogation SNP contains a mixture of the two alleles of the neighboring SNP within the same feature.

In many aspects samples are prepared for hybridization by amplification, fragmentation of the amplicons and labeling of the fragments. The fragments are hybridized to the array under conditions that facilitate allele specific hybridization. Unhybridized material is removed and the array is analyzed to obtain a hybridization pattern. The hybridization pattern is analyzed, preferably using a computer system, to determine genotypes or sequence of the mtDNA and genomic DNA. In preferred embodiments the methods may be used for high-throughput analysis of samples containing mitochondrial DNA or nuclear DNA where the DNA may be partially or substantially degraded.

In one aspect the hybridization pattern that is obtained is compared to additional hybridization patterns from known sources. If the unknown sample is from an individual whose identity is suspected, the hybridization pattern may be compared to individuals that are maternally related to the suspected individual or to a sample known to be from the suspected individual. If the patterns meet a minimum threshold of similarity the identity of the individual may be confirmed. The threshold will vary depending on the number of pieces of information obtained and the type of information.

In one aspect amplified, fragmented, biotin-labeled target is injected into microarrays embedded in cartridges. Hybridization and detection proceed according to standard methods. For detailed protocols see, for example, Affymetrix Mapping Assay manual and CustomSeq Manual (Affymetrix, Inc.). For ultra-small chips (e.g. 1 mm), hybridization is accomplished by affixing the chips to “pegs” and “dipping” the chips into hybridization solution. Following hybridization, the arrays are washed in a series of increasingly stringent buffers to remove unbound target and to improve specificity. For chips in cartridges, the washing occurs in automated fluidics stations; for ultra-small chips, dipping is employed. Following the wash procedure, chips are scanned and image files are created. Data files corresponding to the type of chip being analyzed are generated by the software in an automated fashion.

In many embodiments samples are processed using high throughput methods. See U.S. patent Publication 20030124539 and WO 03/030526. A High Throughput Array (HTA) system based on Affymetrix′ GeneChip® technology packaged in the 96-well format may be used. This system allows the workflow from processing samples to scanning arrays to be fully automated. The HTA system may include the following components: (1) an automation platform; (2) a GeneChip high throughput array; and (3) an HTA Scanner. In one embodiment an automation platform based on the Beckman FX core system and standard 96-well automation devices that are accessed by the Beckman system may be used. In another aspect an automation system based on the Caliper Sciclone workstation. Affymetrix has developed systems that process 96 samples in a fully automated mode. A system that processes samples in a 384-well format may also be used. An HTA plate comprising, for example, individual arrays placed in each well of a 96 or 384-well plate, may be used. The HTA plate allows for experimental flexibility in that the size of the array and the shape of the well can be optimized to the number of SNPs or amount of reference sequence in an experiment. The well size can be designed from, for example, a size of 6×6 mm active area for oligonucleotide probe features down to a 1×1 mm size or smaller. The plate may be designed so that individual arrays can be affixed to individual well positions on a plate. This “chiplet” design allows for the chiplets to be fabricated to the size of the experiment with a minimum cost of array per well; and allows for flexible content for each plate, i.e., each well of a plate can have different content so that mixed samples can be run through sample processing and matched to the array plate.

A high throughput scanner may be used to analyze the array plates. In one embodiment the scanner is CCD-based, which allows for throughput without any loss of performance in sensitivity and resolution. In a preferred embodiment the optical system is flexible and can deliver either of two fields-of-view: (1) 2.5×2.5 mm, which provides 2.5 micron pixel resolution, which allows for effective scanning down to 10 micron array features, or (2) 1.0×1.0 mm, with 1.0 micron pixel resolution, which allows for effective scanning down to 5 micron array features. Data acquisition is complete in 35-40 minutes for 6×6mm chips in a 96-well format. The scanner also reads 384-well plates, and data acquisition can be accomplished in approximately 15 minutes or less. The scanner is preferably automation ready and can be placed in-line with the automation process, or can be run off-line by an operator.

Genotyping analysis of samples from unknown sources, according to the disclosed methods, may also be used to characterize the source of the sample. SNP based assays for determination of origin and eye color have been developed. See U.S. patent Publication Nos. 20030211486 and 20030171878, for example. SNP based assays are available to determine if a sample has geographic ancestry selected from the following four groups: African, Indo-European, Native American and East Asian. In one embodiment of the presently disclose methods, a method to specify geographical ancestry at higher resolution, i.e. determination of sub-population group, etc., using ancestry informative markers is disclosed. Large numbers of SNPs may be used to identify geographical ancestry at higher resolution and at higher levels of statistical significance. Large numbers of forensics samples may be analyzed to genotype large numbers of SNPs in a high-throughput and cost-effective manner. See, for example, Shriver, et al. (2003), Hum Genet. 112(4): 387-99.

In many embodiments microarrays are disclosed. Advances in synthetic array technology allow larger numbers of oligonucleotides to be synthesized on smaller sized arrays. In one aspect resequencing arrays are designed to include a probe for each of the 4 bases for each nucleotide position in the reference sequence on both strands; the resequencing of the entire human mitochondrial genome on both strands requires about 135,000 probes, see Maitra et al., (2004). In order to capture as much information as possible from the degraded sample, arrays are disclosed that comprise probes for resequencing or genotyping mtDNA, as well as probe sets to interrogate the genotype of several thousand SNPs. The nuclear SNP probes sets may be used to determine the extent of nuclear DNA remaining in the sample that can be amplified. Such a chip, containing both nuclear DNA SNPs and mitochondrial DNA probes may be, for example, about 6.75 mm in size, small enough to be processed in 96-well format for subsequent ultra-high-throughput (HTA) applications. In some embodiments an array comprising probe sets for less than 1000 nuclear SNPs is disclosed. This array may be less than 6.75 mm in size. The number of features present on arrays depends on the feature size and the array size. Arrays may be synthesized in a variety of formats, for example, 49, 400, 1600 or 5000 format. Format is determined by the size of the array, for example the 49 format is larger than the 400 format so with 8 um features a 49 format array includes approximately 2,600,000 features and a 400 format array includes approximately 100,000 features. Varying format and feature size is shown in Table 1.

TABLE 1

Feature size

| Format | 18 um | 11 um | 8 um | 5 um |
|---|---|---|---|---|
| 49 | 500000 | 1400000 | 2600000 | 6500000 |
| 400 | 20000 | 110000 | 100000 | 500000 |
| 1600 | 4900 | 13000 | 25000 | 64000 |
| 5000 | 3000 | 8000 | 16000 | 40000 |

In some embodiments analysis of samples is done in a low-cost high-throughput platform configuration. The HTA platform described above is available to implement high-throughput analysis of degraded samples. Following assay optimization as described below, the MCP sample preparation method will be adapted to the current platform. This will require modifying the upfront target preparations steps, which are currently configured for expression analysis. The downstream steps of hybridization, washing and scanning will be very similar to those already in place for expression arrays.

Genotyping and resequencing algorithms have been validated extensively to give high accuracy on samples with complete or near complete data. For example, the 10K and 100K chips routinely make genotype calls on >95% of the SNPs on the array. Lower call rates are expected when samples of degraded DNA are used because some of the SNPs will not be amplified. Other SNPs that reside in fragments that are not degraded will still be amplified. In a preferred embodiment call rates less than 90% may be observed with high accuracy. Resequencing algorithms that are modified to take into account samples from which only a fraction of the total genetic material is amplified are used. Rather than assuming complete amplification, the algorithm is trained to recognize partial data and make high accuracy genotype and sequence base calls accordingly. Because of the large amount of content available on the microarrays, recovering even a portion of the available information from the target, for example less than 75%, 50%, 25% or 10% of the polymorphic positions interrogated by the array, may be used for forensic applications, for example in identifying human remains.

### CONCLUSION

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

1. A method of characterizing an unknown sample comprising:
obtaining nucleic acid from the sample;
fragmenting the nucleic acid with a restriction enzyme;
ligating an adaptor to at least some of the fragments to generate adapter-ligated fragments, wherein the adaptor has a single stranded overhang that is complementary to the single stranded overhang generated by the restriction enzyme;
amplifying at least some of the adapter-ligated fragments by polymerase chain reaction using a primer complementary to the adaptor to generate amplified fragments;
labeling the amplified fragments;
hybridizing the amplified fragments to an array of probes wherein the array of probes comprises at least 10,000 different sequence probes, wherein each of the at least 10,000 different sequence probes is complementary to mitochondrial DNA sequence and each of the different sequence probes is present in a different feature of the array; and, detecting a hybridization pattern wherein the hybridization pattern is characteristic of the unknown sample.

2. The method of claim 1 wherein the array of probes further comprises a plurality of genotyping probe sets, wherein a genotyping probe set comprises a first allele specific probe for a first allele of a biallelic human nuclear SNP and a second allele specific probe for a second allele of said biallelic human nuclear SNP.

3. The method of claim 2 wherein the array of probes comprises 1,000 genotyping probe sets.

4. The method of claim 3 wherein the array of probes comprises resequencing probe sets for interrogating the sequence of 2 kilobases of human mitochondrial DNA, wherein a resequencing probe set comprises four probes that are a perfect match to the sequence on either side of the interrogation position, each of the four probes containing a different base, A, G, C or T, at the interrogation position.

5. The method of claim 4 wherein the 2 kilobases of human mitochondrial DNA is non-contiguous.

6. The method of claim 1 wherein the restriction enzyme has a recognition site consisting of 4 base pairs.

7. The method of claim 1 wherein the unknown sample is from a first individual and the hybridization pattern that is characteristic of the unknown sample is compared to a second hybridization pattern, wherein the second hybridization pattern is characteristic of a sample from a known individual suspected of being related to the first individual, and further comprising making a determination that the first individual is related to the second individual if the hybridization patterns meet a threshold level of similarity.

8. The method of claim 1 wherein the sample is fragmented with 2 restriction enzymes and wherein each of the restriction enzymes has a four base pair recognition sequence and each cleaves DNA to generate a single stranded overhang.

9. The method of claim 8 wherein different adaptor sequences are ligated to the different overhangs so that the ends of the adaptor ligated fragments are not self complementary.

10. An array of probes comprising 10,000 probes for resequencing mitochondrial DNA and 1,000 probes for genotyping nuclear single nuclear polymorphisms wherein each probe is present in a different feature of the array.

11. The array of claim 10 wherein the array comprises a probe set to interrogate each of at least 1,000 different biallelic human nuclear SNPs.

12. The array of claim 11 wherein at least 100 of the SNPs are ancestry informative markers.

13. The array of claim 12 wherein the array comprises resequencing probe sets for 2 kilobases of human mitochondrial DNA and genotyping probe sets for each of at least 10,000 human nuclear SNPs, wherein a genotyping probe set comprises a first allele specific probe for a first allele of a biallelic human nuclear SNP and a second allele specific probe for a second allele of said biallelic human nuclear SNP and wherein a resequencing probe set comprises four probes that are a perfect match to the sequence on either side of the interrogation position, each of the four probes containing a different base, A, G, C or T, at the interrogation position.

14. An array comprising a plurality of genotyping probe sets to interrogate human mitochondrial polymorphisms and a plurality of genotyping probe sets to interrogate human nuclear polymorphisms.

15. The array of claim 14 wherein the array interrogates 1,000 human mitochondrial polymorphisms and 500 human nuclear polymorphisms.


SEQUENCE LISTING (25 sequences)

SEQ ID NO: 1 - DNA, 25 bp, Homo Sapien
acgttatatagatgccatgctatag

SEQ ID NO: 2 - DNA, 25 bp, Homo Sapien
acgttatatagaggccatgctatag

SEQ ID NO: 3 - DNA, 25 bp, Homo Sapien
acgttatatagacgccatgctatag

SEQ ID NO: 4 - DNA, 25 bp, Homo Sapien
acgttatatagaagccatgctatag

SEQ ID NO: 5 - DNA, 25 bp, Homo Sapien
cgttatatagaatccatgctatagt

SEQ ID NO: 6 - DNA, 25 bp, Homo Sapien
cgttatatagaagccatgctatagt

SEQ ID NO: 7 - DNA, 25 bp, Homo Sapien
cgttatatagaacccatgctatagt

SEQ ID NO: 8 - DNA, 25 bp, Homo Sapien
cgttatatagaaaccatgctatagt

SEQ ID NO: 9 - DNA, 25 bp, Homo Sapien
gttatatagaagtcatgctatagta

SEQ ID NO: 10 - DNA, 25 bp, Homo Sapien
gttatatagaaggcatgctatagta

SEQ ID NO: 11 - DNA, 25 bp, Homo Sapien
gttatatagaagccatgctatagta

SEQ ID NO: 12 - DNA, 25 bp, Homo Sapien
gttatatagaagacatgctatagta

SEQ ID NO: 13 - DNA, 25 bp, Homo Sapien
ttatatagaagctatgctatagtac

SEQ ID NO: 14 - DNA, 25 bp, Homo Sapien
ttatatagaagcgatgctatagtac

SEQ ID NO: 15 - DNA, 25 bp, Homo Sapien
ttatatagaagccatgctatagtac

SEQ ID NO: 16 - DNA, 25 bp, Homo Sapien
ttatatagaagcaatgctatagtac

SEQ ID NO: 17 - DNA, 21 bp, Homo Sapien
  misc_feature (1)..(6): n is a, c, g, or t
nnnnnngnnnmnnnnnnnnnn

SEQ ID NO: 18 - DNA, 25 bp, Homo Sapien
  misc_feature (1)..(8): n is a, c, g, or t
nnnnnnnncnnntnnnnnnnnnnnn

SEQ ID NO: 19 - DNA, 25 bp, Homo Sapien
  misc_feature (1)..(8): n is a, c, g, or t
nnnnnnnncnnnannnnnnnnnnnn

SEQ ID NO: 20 - DNA, 25 bp, Homo Sapien
  misc_feature (1)..(8): n is a, c, g, or t
nnnnnnnncnnngnnnnnnnnnnnn

SEQ ID NO: 21 - DNA, 25 bp, Homo Sapien
  misc_feature (1)..(8): n is a, c, g, or t
nnnnnnnncnnncnnnnnnnnnnnn

SEQ ID NO: 22 - DNA, 25 bp, Homo Sapien
  misc_feature (1)..(12): n is a, c, g, or t
nnnnnnnnnnnncnnntnnnnnnnn

SEQ ID NO: 23 - DNA, 25 bp, Homo Sapien
  misc_feature (1)..(12): n is a, c, g, or t
nnnnnnnnnnnngnnntnnnnnnnn

SEQ ID NO: 24 - DNA, 25 bp, Homo Sapien
  misc_feature (1)..(12): n is a, c, g, or t
nnnnnnnnnnnncnnngnnnnnnnn

SEQ ID NO: 25 - DNA, 25 bp, Homo Sapien
  misc_feature (1)..(12): n is a, c, g, or t
nnnnnnnnnnnngnnngnnnnnnnn